(19) United States
(12) Patent Application Publication
(10) Pub. No.: US 2024/0285431 A1
(43) Pub. Date: Aug. 29, 2024

(54) ELECTROSTATIC VITREORETINAL BRUSH

(71) Applicant: OPTICGON LLC, Flushing, NY (US)

(72) Inventor: Ian H. CHAN, Flushing, NY (US)

(21) Appl. No.: 18/588,662

(22) Filed: Feb. 27, 2024

Related U.S. Application Data
(60) Provisional application No. 63/448,588, filed on Feb. 27, 2023.

(51) Int. Cl. A61F 9/00

(52) U.S. Cl. CPC A61F 9/0026

(57) ABSTRACT

This disclosure provides an ophthalmological surgical device to perform amniotic membrane transplantation or internal limiting membrane transplantation to the macula of an eye of a subject. The device includes a handle portion; a rod-shaped body attached to one end of the handle portion; a brush portion made of materials capable of holding electrostatic charges attached to the distal end of the rod-shaped body. Making use of the triboelectric effect in static electricity, the brush will attract biological membranes such as the amniotic membrane and allow placement of the membrane over the macula or other desired retinal position with precision and ease.

### FIELD

This disclosure relates to the field of ophthalmic surgical instruments for treating macular injuries or diseases. In particular, this disclosure relates to devices and methods for transplanting amniotic membranes to the macula of the eye of a subject employing the principle of selective electrostatic attraction of a device to the amniotic membrane or internal limiting membrane.

### RELATED APPLICATIONS

This disclosure claims priority to U.S. Provisional Application No. 63/448,588, filed Feb. 27, 2023, the contents of which are herein incorporated by reference.

### BACKGROUND

One element of vitreoretinal surgery is to place biological or synthetic membranes over the macula region to repair complex and large macular holes Placing a membrane such as an amniotic membrane graft over the macula hole requires a great amount of precision and needs to be performed without injury or harm to the neurosensory macula. Intraocular forceps, soft tips, scrapers, wire loops, erasers and picks have been previously used for placing and positioning of membranes over the macula, but each involves a risk of unintentional damage to the macula.

Triboelectrification, or contact electrification, describes the transmission of electrons across surfaces when there is physical contact. Triboelectrification is the physical transport of electrons between two materials. The difference in the electron affinities of the two materials determines which the direction of electron transfer, where the material with a higher electron affinity will attract electrons from the material with a lower electron affinity. The donor and acceptor materials are typically identified by their order in the triboelectric series.

All documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

Citation or identification of any document in this application is not an admission that such document is available as prior art.

### SUMMARY

The embodiments described herein have many attributes and aspects including, but not limited to, those set forth or described or referenced in this Summary. It is not intended to be all-inclusive and the embodiments described herein are not limited to or by the features or embodiments identified in this Summary, which is included for purposes of illustration only and not restriction.

In one aspect, this disclosure relates to a surgical instrument used to perform amniotic membrane transplantation to the macula of the eye of a subject.

In some aspects, the disclosure related to a surgical instrument used to perform internal limiting membrane transplantation to the macula of the eye of a subject.

In some aspects, this disclosure relates to a vitreoretinal brush used for ophthalmic surgery, comprising: a handle portion; a rod shaped body attached to one end of said handle portion; and a brush tip fitted along a direction toward a front end of said rod-shaped body to the front end side; wherein the brush tip comprises one or a plurality of filaments made of a material in the negative triboelectric spectrum. The one or a plurality of filaments can comprise negative electrostatic charges, enhancing its attraction for biological or artificial membranes

In some aspects, the material in the negative triboelectric spectrum can be selected from: Teflon (polytetrafluoroethylene), silicone (polydimethylsiloxane), rubber, vinyl, Acetal, viton, garolite, polyester, polystyrene, or combinations thereof.

In some aspects, the circumferential dimension of the instrument can be 23-29 gauge in size.

In some aspects, the brush tip and rod-shaped body can be surrounded by a retractable metallic sleeve where the retraction is controlled by a sliding knob on the body of the instrument.

In some aspects, this disclosure relates to a method of translocating an amniotic membrane, the method comprising:
a. charging a vitreoretinal brush filament of any of claims 1-5 by rubbing the brush tip against a material on the positive or neutral end of the triboelectric spectrum to produce a charged vitreoretinal brush filament;
b. loading the brush tip with an amniotic membrane or internal limiting membrane or portion thereof by contacting said charged vitreoretinal brush filament with the choroidal side of an amniotic membrane, internal limiting membrane or portion thereof to form a brush-membrane complex comprising an amniotic membrane end;
c. introducing a trocar into the eye of a subject;
d. translocating the brush-membrane complex through the trocar;
e. contacting the amniotic membrane end of the brush-membrane complex to a macula in said eye of a subject; and
f. releasing said amniotic membrane or internal limiting membrane from said brush-membrane complex such that the choroid side of the amniotic membrane selectively adheres to the macula.
   In some aspects, the brush tip and rod-shaped body can be surrounded by a retractable metallic sleeve, and the method further comprising the steps of:
i. surrounding the brush-membrane complex with the retractable metallic sleeve before entry through the trocar (e.g., before step (d) is performed); and
ii. retracting the retractable metallic sleeve from the brush-membrane complex after entry through the trocar (e.g., after step (d) is performed).

### BRIEF DESCRIPTION OF THE DRAWINGS

The drawings form part of the present specification and are included to further demonstrate certain aspects of the embodiments described herein. These embodiments may be better understood by reference to one or more of the following drawings in combination with the detailed description.

FIG. 1 depicts a side view of the electrostatic vitreoretinal brush tool of one embodiment according to the present invention; the tool comprises a handle (103), a metallic shaft (102), and a brush-tip (101). The brush-tip (101) comprises one or a plurality of flexible filaments comprised of a triboelectric material with high negative charges retention capability.

FIG. 2 is a perspective and enlarged view of the brush tip (101) and shaft (102). The instrument can be sized and fit into a trocar system having a size ranging from 20 to 30 gauge (and in some embodiments, 23, 25, 27 or 29 gauge trocar systems).

FIG. 3 is a perspective and enlarged view of one embodiment of a spatula-like brush tip (301), and shaft (302).

FIG. 4 is a perspective and enlarged view of one embodiment of a brush tip (402) connected to a round shaft (403). This brush tip contains fewer brush (402) filaments and contains a retractable outer metallic sleeve (401) which protects the contents of the brush filaments and amniotic membrane or internal limiting membrane during instrument insertion through the trocar. The configuration depicted in FIG. 4 shows the outer sleeve (401) in a first position (extended) which covers the brush tip (402).

FIG. 5 is a perspective and enlarged view of the same instrument in FIG. 4, in a second configuration where the outer sleeve (401) is retracted, exposing the brush filaments (402).

FIG. 6A depicts one embodiment of the brush tool wherein the outer sleeve (401) is connected to a slider knob (603) on the handle (103) and is a configuration where the outer sleeve extends over the brush tip portion.

FIG. 6B depicts one embodiment of the brush tool wherein the outer sleeve is in a configuration where the outer sleeve is retracted to as to expose the brush tip portion.

### DETAILED DESCRIPTION

### Certain Definitions

Unless defined otherwise, all technical and scientific terms used herein have the same meaning as commonly understood by one of ordinary skill in the art to which this disclosure is related.

Units, prefixes, and symbols are denoted in their System International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The use of the terms “a” and “an” and “the” and similar referents are to be construed to cover both the singular and the plural, unless otherwise indicated herein or clearly contradicted by context.

As used herein, the term “about” means ±10%. For example, the term “about 10 millimeters” refers to a range of 9 to 11 millimeters.

A “handle” is the part of a device designed to be held or manipulated by a human hand.

As used herein, the term “subject” as used herein refers to humans, higher non-human primates, rabbits, rodents, domestic, cows, horses, pigs, sheep, dogs and cats. In one embodiment, the subject is a human.

As used herein, the terms “treat” and “treatment” refer to both therapeutic treatment and prophylactic or preventative measures, wherein the object is to prevent or decrease an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder and those prone to have the condition or disorder or those in which the condition or disorder is to be prevented.

As described herein, any concentration range, percentage range, ratio range or integer range is to be understood to include the value of any integer within the recited range and, when appropriate, fractions thereof (including one-tenth and one-hundredth of an integer), unless otherwise indicated.

### Certain Methods

An amniotic membrane is often used as a healing scaffold to diseased or injured macula. Amniotic membranes comprise a chorionic (sticky) side and a hydrophobic (non-sticky) side. During amniotic membrane transplantation, the amniotic membrane must be placed with the chorionic or sticky side to the retinal pigment epithelium (RPE) layer. Surgeons need to make sure that the membrane does not flip over resulting in placement of the incorrect side of the membrane to the RPE. The sticky chorionic side of the amniotic membrane may often curl up and adhere to itself during tissue placement, which presents a surgical challenge as it needs to be unfolded.

An internal limiting membrane (ILM) fragment is often produced during the ILM peeling process in a routine macular hole repair surgery. The ILM can serve as a healing scaffold where the ILM is laid over the macular hole or as a “plug” to fill the hole (Zhang, L., et al. Internal limiting membrane insertion technique combined with nerve growth factor injection for large macular hole. BMC Ophthalmol 19, 247 (2019). doi.org/10.1186/s12886-019-1258-z). Due to the thin nature of the ILM fragments, the ILM fragments can be lost or displaced during its placement process.

The inventors have recognized that the triboelectric effect may be employed to charge an amniotic membrane handling device with an electrostatic charge. In some embodiments, the amniotic membrane handling device is a vitreoretinal brush. The vitreoretinal brush comprises a tip with a plurality of filaments, wherein the filaments are of a material of a high negative value on the triboelectric scale. In some embodiments, during a surgical procedure, the brush tip can be charged by rubbing the brush tip against a material such as surgical cotton to form an electrostatic charge on the tip material. Then vitreoretinal brush can be used to attract the amniotic side (non-sticky) side of the amniotic membrane. The amniotic membrane can then be introduced into the eye via a trocar. Because the chorionic side of the amniotic membrane is sticky, it can be brushed onto the RPE of the open macula hole. The brush tip is also a convenient tool to flip over the amniotic membrane graft if needed and to adjust its position on the macula.

In some embodiments, the electrostatic force between the brush tip and the membrane is relatively weak such that the brush tip can maintain the attraction to the membrane for about 11 minutes or less. Brushing the membrane against the retina with the sticky side facing the retina will release the membrane, resulting in the placement of the membrane to the retina.

In some embodiments, the charging step, the step of rubbing the brush tip against a material such as surgical cotton, can be performed in a controlled humidity environment to prevent the premature discharge of any built up triboelectric charge.

In some embodiments, the material of the brush tip and the material against which the brush tip is rubbed (“charging surface”) to charge said brush tip are different on the triboelectric spectrum. The brush tip material and the charging surface can be on the same side of the triboelectric spectrum or different sides of the triboelectric spectrum. When the brush tip material and the charging surface are on the same side of the triboelectric spectrum, the two materials are selected to have different triboelectric effects, such that the rubbing the materials will result in the buildup of a triboelectric charge (a “static charge”.)

According to the ophthalmic treatment tool with the construction mentioned above, the methods and devices of this disclosure allow for the placement and positioning of a biological membrane over the open macular hole in a facile and precise manner. In some embodiments, the biological membrane is selected from an amniotic membrane, internal limiting membrane or a synthetic membrane. In some embodiments, the synthetic membrane is a hydrogel material. Risk of causing inadvertent retinal damage is reduced by reducing the need to manipulate the membrane to be implanted inside the eye. The brush also minimizes trauma to the macula and is an ideal tool to adjust the position of the membrane over delicate tissues such as the macula, in contradistinction to the case of the conventional instruments such as forceps, scrapers and wire loops. Depending on the surgical technique, the biological membrane can be placed in a subretinal space under the macula, as a plug to fill the space of the macula hole, or in epi-macular location.

Various other objects, features and attendant advantages of the present invention will be more fully appreciated as the same becomes better understood from the following detailed description when considered in connection with the accompanying drawings in which like reference characters designate like or corresponding parts throughout the several views.

### Certain Devices

In some embodiments, this disclosure provides for a vitreoretinal brush which comprises: a handle portion; a rod-shaped body attached to one end of the handle portion; a brush portion comprising one or a plurality of filaments, wherein the filaments comprise a materials on the negative charge of the triboelectric spectrum. The triboelectric spectrum is the ability of a bulk material to retain an induced charge, and is measured as “charge affinity” nC/J (nano ampsec/wattsec of friction). In some embodiments, the negative triboelectric material can be: Teflon (Polytetrafluoroethylene), Viton (dipolymers comprised of hexafluoropropylene and vinylidene fluoride), silicone, vinyl, Acetal (polyacetal, polyoxymethylene), garolite (glass epoxy fiberglass laminate), polyester, polystyrene, polyvinylidene fluoride (PVDF), PVDF copolymers, polydimethylsiloxane (PDMS), PVC (polyvinylchloride), polymethylmethacrylate, polytetrafluoroethylene, or poly(methylmethacrylate)-copoly(1H-1H-perfluoroctylmethyacylate), Synthetic Rubber, cellulose acetate, Rayon (viscose, cellulose made using alkali and carbon disulfide), Polyester, Celluloid (nitrocellulose composition), Polystyrene, Orlon (polyacrylonitrile copolymers), Acrylic, Cellophane Tape, Polyvinylidene chloride (Saran), Polyurethane, Polyethylene, Polypropylene, Polyvinylchloride (PVC), or Kel-F (Polychlorotrifluoroethylene). In some embodiments, the brush is fitted along a direction toward a front end of the rod-shaped body.

In some embodiments, the diameter of the vitreoretinal brush is configured to be sufficiently small to traverse through a vitrectomy trocar system. In some embodiments, the trocar is a 19-30 gauge range. In some embodiments, the size of the trocar is 19, 20, 21, 22, 23, 24, 25, 26, 27, 28, 29, or 30 gauge, or a range between any of the aforementioned values.

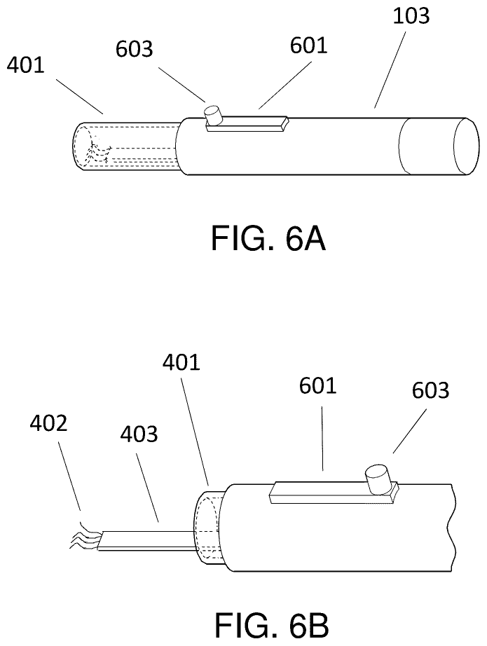

In one embodiment, the brush tip and shaft can be surrounded by a retractable sleeve during the instrument insertion. The retractable sleeve can protect both the brush filaments and the amniotic (or synthetic) membrane “loaded” on the brush filaments with the chorionic side on the opposite side of the brush tip. As shown in FIG. 6, in some embodiments, the retractable sleeve can be retracted with a sliding knob on the instrument body after instrument insertion into the trocar.

### Description of the Preferred Embodiment

The invention will now be more specifically described with reference to embodied configurations illustrated in the drawings attached hereto, but the present invention should not be restricted to those embodied configurations.

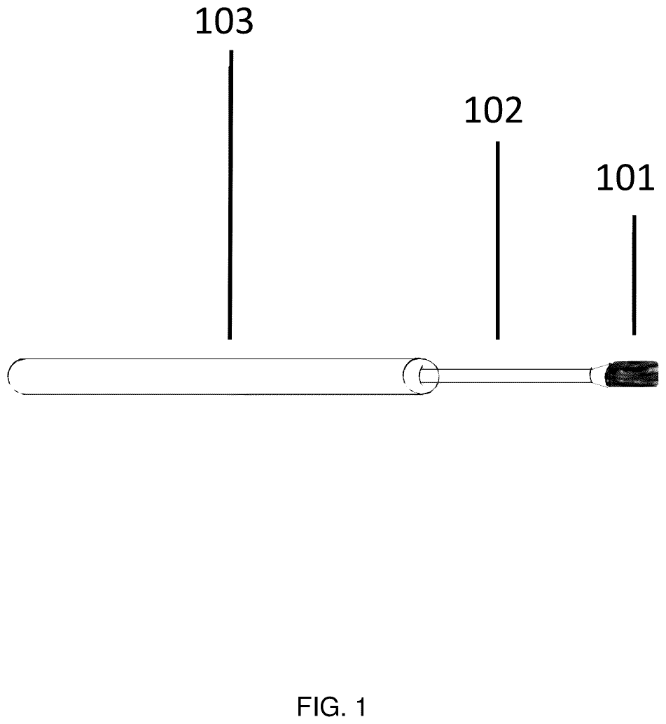

Referring to FIG. 1, which depicts a side view of an ophthalmic treatment tool of one embodiment according to the present invention. An ophthalmic treatment tool of the present invention, as shown in the figures, is characterized by having a handle (103) a rod-shaped body or shaft (102) attached to one end of the handle (103), a brush tip (101) fitted along a direction toward a front end of the rod-shaped body or shaft (102).

The handle (103) can be well grasped by user's fingers for a surgery, which is not restricted to the example shown in the figure and may have any shape unless it troubles the users in their operation. In the case of grasp by the fingers, the handle (103) is suitably formed to be mostly as thick as e.g., pencils, pens, or chopsticks which the users are generally used to handling. With respect to materials, the handle (103) is made from material which have no hygienic problem, the material being, for example, wood, metal, plastic or the like, the plastic being preferably used for the handle, the better of metals being silver, stainless steel, titanium or the like, the titanium being preferably used for the handle in the light of the strength, lightweight and/or others.

One end of the handle is fixed to a rod-shaped body (102) in a certain way that may be modified as needed. The rod-shaped body (102) is provided for purposes of supporting a brush tip (101) which is placed on the front end side of the rod-shaped body (102). In some embodiments, the rod-shape body (102) is made from titanium or other conductive, and surgically appropriate, metal. The rod-shape body is of a material that will not discharge the triboelectric charge built up on the brush tip.

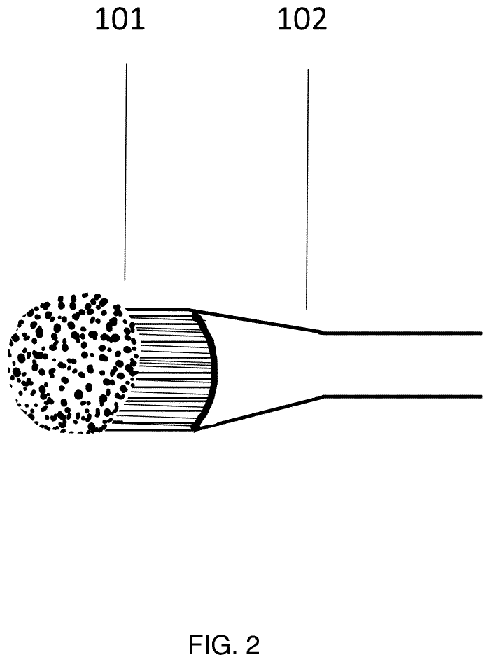

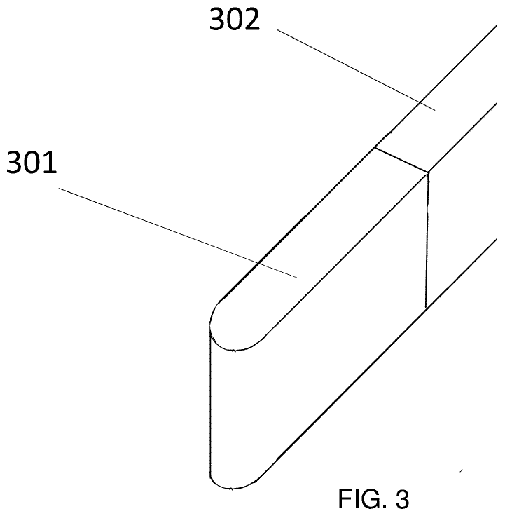

An end portion of the rod-shaped body (102) is provided with the brush tip (101). In one embodiment (as shown in FIG. 2), the brush tip (101) is made of small filaments made of Teflon which is attached to the rod-shaped body (102). In another embodiment (FIG. 3), the brush tip (101) is composed of a spatula-like brush made of silicone or rubber. Both the brush filament tip and the spatula tip will be sufficiently pliable and flexible so as not to carry the risk of causing damage to the retina and so on when placing or positioning the biological or artificial membranes. In the ideal method of usage, the brush tips will be rubbed against sterile medical cotton to create electrostatic charges on the brush tip by taking advantage of the triboelectric effect of the brush material. The human amniotic membrane consists of a chorionic side which is sticky and an amniotic side which is non-sticky. The goal of the surgery is to place the chorionic or sticky side onto the open macular hole. The tip of the electrostatic vitreoretinal brush will attract the anionic or non-sticky side of the amniotic membrane. The paper backing of the chorionic side is then removed so that the chorionic side is facing away from the brush tip. The instrument with the amniotic membrane is then inserted through the trocar. The membrane is then placed over the macular by gently brushing the membrane off the instrument at the macula. Because the chorionic side of the amniotic membrane is sticky, it will adhere to the RPE layer of the open macular hole and overcome the weaker electrostatic attraction on the opposite side between the brush and the amniotic side of the amniotic membrane.

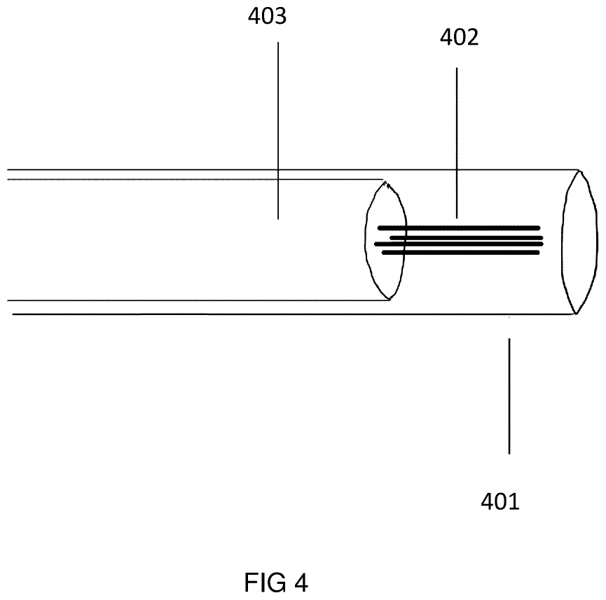

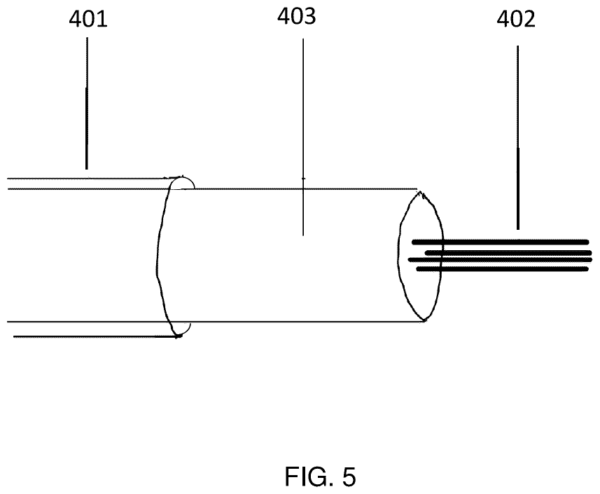

In some embodiments, the brush tip (101) can be further modified to contain a retractable outer sleeve as shown in FIG. 4, FIG. 5, FIG. 6A, and FIG. 6B. The metallic outer sleeve can be in closed position during instrument insertion via the trocar, thus protecting the brush tip, brush filaments and the amniotic membrane adherent to the filaments. Once inside the eye, the outer sleeve can be retracted and expose the brush tip and the amniotic membrane adherent to the brush tip. The amniotic (or synthetic) membrane can be transferred from the brush tip to the macula region by a gentle brushing motion. The retractable sleeve (401) can be controlled by a slider knob (603) on the handle (103) of the instrument as shown in FIG. 6. The slider knob (603) can be configured to be connected to a slideable portion (601) which is connected to the retractable sleeve (401). Similarly, an internal limiting membrane fragment can be loaded onto the brush filament tips and transferred from the brush tip (402) to the macula by gentle brushing motion.

The retractable sleeve can be made of any surgically suitable metal that does not prematurely discharge the triboelectric charge built up on the brush tip.

In some embodiments, the brush tip comprises a plurality of filaments. The filaments are made of a triboelectric material. The number of filaments can range from 1 to 10,000 and be of a diameter ranging from 0.05 microns to 3 mm. The length of the filaments can range from 1 mm to 20 mm, or longer. The length of the filament can be selected based on the rigidity of the filament material such that the position of the “loaded” membrane can be spatially controlled.

In some embodiments, the components of the instrument are selected to minimize premature discharge of any built-up triboelectric charge on the brush tip. The filaments of the brush tip can be electrically isolated from the rod shaped body. The rod shaped body can be electrically isolated from the handle. The handle can be electrically isolated from the hand of the user. The electrical isolation can be performed by the inclusion of an insulating material between any of the aforementioned components of the instrument (and/or the user's hand).

The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the technology and does not pose a limitation on the scope of the technology unless otherwise claimed. No language in the specification should be construed as indicating any non-claimed element as essential to the practice of the technology.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the embodiment.

Embodiments are described herein, including the best mode known to the inventors. Variations of those embodiments may become apparent to those of ordinary skill in the art upon reading the foregoing description. The inventors expect skilled artisans to employ such variations as appropriate, and the inventors intend for the embodiments to be practiced otherwise than as specifically described herein. Accordingly, this technology includes all modifications and equivalents of the subject matter recited in the claims appended hereto as permitted by applicable law. Moreover, any combination of the above-described elements in all possible variations thereof is encompassed by embodiments unless otherwise indicated herein or otherwise clearly contradicted by context.

The preceding merely illustrates the principles of various embodiments of the disclosure. It will thus be appreciated that those skilled in the art will be able to devise various arrangements which, although not explicitly described or shown herein, embody the principles of the invention and are included within its spirit and scope. Furthermore, all examples and conditional language recited herein are principally intended expressly to be only for pedagogical purposes and to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents and equivalents developed in the future, i.e., any elements developed that perform the same function, regardless of structure.

All numbers expressing quantities or parameters used in the specification are to be understood as additionally being modified in all instances by the term “about”. Notwithstanding that the numerical ranges and parameters set forth, the broad scope of the patient matter presented herein are approximations, the numerical values set forth are indicated as precisely as possible. For example, any numerical value may inherently contain certain errors, evidenced by the standard deviation associated with their respective measurement techniques, or round-off errors and inaccuracies.

What is claimed is:

1. A vitreoretinal brush instrument for ophthalmic surgery, comprising:
a. a handle portion;
b. a rod shaped body attached to one end of said handle portion; and
c. a brush tip fitted along a direction toward a front end of said rod-shaped body to the front end side;
wherein said brush tip comprises one or a plurality of filaments made of a material in the negative triboelectric spectrum.

2. The vitreoretinal brush instrument of claim 1, wherein the one or a plurality of filaments comprise negative electrostatic charges, enhancing its attraction for biological or artificial membranes.

3. The vitreoretinal brush instrument of claim 1, wherein the material in the negative triboelectric spectrum is selected from: Teflon (polytetrafluoroethylene), silicone (polydimethylsiloxane), rubber, vinyl, Acetal, viton, garolite, polyester, or polystyrene.

4. The vitreoretinal brush instrument of claim 1, wherein the circumferential dimension of the rod-shaped body is 23 to 29 gauge in size.

5. The vitreoretinal brush instrument of claim 1, wherein the brush tip and rod-shaped body is surrounded by a retractable metallic sleeve where the retraction is controlled by a sliding knob on the body of the instrument.

6. A method of translocating an amniotic membrane or internal limiting membrane, the method comprising:
a. charging the brush tip of a vitreoretinal brush instrument of any of claims 1-5 by rubbing the brush tip against a material on the positive or neutral end of the triboelectric spectrum to produce a charged vitreoretinal brush filament;
b. loading the brush tip with an amniotic membrane or internal limiting membrane or portion thereof by contacting said charged vitreoretinal brush filament with the choroidal side of an amniotic membrane, internal limiting membrane or portion thereof to form a brush-membrane complex comprising an amniotic membrane end;
c. introducing a trocar into the eye of a subject;
d. translocating the brush-membrane complex through the trocar;
e. contacting the amniotic membrane end of the brush-membrane complex to a macula in said eye of a subject; and
f. releasing said amniotic membrane or internal limiting membrane from said brush-membrane complex such that the choroid side of the amniotic membrane selectively adheres to the macula.

7. The method of claim 6, wherein the brush tip and rod-shaped body is surrounded by a retractable metallic sleeve, and the method further comprising the steps of:
i. surrounding the brush-membrane complex with the retractable metallic sleeve before performing step (d) translocating the brush-membrane complex through the trocar; and
ii. retracting the retractable metallic sleeve from the brush-membrane complex after performing step (d).
